(19) United States
(12) Patent Application Publication
(10) Pub. No.: US 2010/0092972 A1
(43) Pub. Date: Apr. 15, 2010

(54) ASSAY FOR GENE EXPRESSION

(75) Inventors: Douglas Spencer Millar, Revesby (AU); John R. Melki, Dolls Point (AU); Geoffrey W. Grigg, Linley Point (AU)

Correspondence Address: KNOBBE MARTENS OLSON & BEAR LLP, 2040 MAIN STREET, FOURTEENTH FLOOR, IRVINE, CA, 92614, US

(73) Assignee: HUMAN GENETIC SIGNATURES PTY LTD., North Ryde, NSW (AU)

(21) Appl. No.: 12/531,482

(22) PCT Filed: Mar. 14, 2008
(86) PCT No.: PCT/AU2008/000367

§ 371 Date: Nov. 16, 2009

(30) Foreign Application Priority Data
Mar. 16, 2007 (AU) 2007901397

(51) Int. Cl. C12Q 1/68

(52) U.S. Cl. 435/6

(57) ABSTRACT

An assay for gene expression comprising treating RNA with an agent such as bisulphate that substantially removes secondary structure of the RNA; and measuring the presence or amount of treated RNA so as to obtain an indication of gene expression. The invention also includes use of oligonucleotide, PNA, LNA or INA probes in the assay.

### TECHNICAL FIELD

The present invention relates to assays for gene expression which do not necessarily require conversion of RNA to DNA.

### BACKGROUND ART

Methods presently used for estimating gene expression by measurement of RNA output in a population of cells, such as microarray expression profiling using chips (Schena et al, 1995, Science 270:467-470; Chee et al, 1996, Science 274:610-614) or via serial analysis of gene expression SAGE (Velculescu et al, 1995, Science 270:484-487), or via total gene expression analysis TOGA (Sutcliffe et al, 2000, Proc Natl Acad Sci USA 97:1976-1981), or via randomly ordered addressable high density optical sensor arrays (Michael et al, 1998, Anal Chem 70:1242-1248), or via massively parallel signature sequencing MPSS on microbead arrays (Brenner et al, 2000, Nature Biotechnology 18:630-634) may not necessarily provide accurate information on the true extent or amount of gene expression. Many of the methods used are indirect as they first require reverse transcription mediated conversion of RNA to corresponding cDNA molecules and then amplification and labelling of the cDNA population. At best, such current methods provide only an indication of gene expression but do not provide an accurate measurement of expression of a particular gene of interest.

These difficulties are now becoming very apparent. For example, in the SAGE technology biases have been described (Stollberg et al, 2000, Genome Research 10:1241-1248), in chips (Chudin et al, 2001, Gene Biology 3:0005.1-0005.10; Kothapalli et al, 2002, BMC Bioinformatics 3:1-10; Workman et al, 2002, Genome Biology 3:0048.1-0048.16. More generally, problems in current methods have been described (Martin and Pardee, 2000, Proc Natl Acad Sci USA 97:3789-3791; Wang et al, 2000, Proc Natl Acad Sci USA 97:4162-4167).

It is difficult to assay RNA by direct hybridisation with an appropriate specific probe because of the stable secondary structures which form spontaneously and rapidly in an erstwhile denatured RNA molecule.

The present inventors have now developed an improved assay which is capable of providing a more accurate estimate of gene expression in an organism, cell population or tissue sample.

### DISCLOSURE OF INVENTION

In a first aspect, the present invention provides an assay for gene expression comprising:

(a) treating RNA with an agent that substantially removes secondary structure of the RNA; and
(b) measuring the presence or amount of treated RNA so as to obtain an indication of gene expression.

In a second aspect, the present invention provides use of an agent which substantially removes secondary structure of RNA and stabilizes the RNA in assays to estimate or measure gene expression.

In a third aspect, the present invention provides use of probes having selected chemical composition for assaying for gene expression via RNA detection.

Preferably, the probes are composed substantially of bases A (adenine), T (thymine) and C (cytosine) and do not contain significant amounts of G (guanine). Preferably, the probes are substantially free of G (guanine).

The invention also covers use of oligonucleotide, PNA, LNA or INA probes in an assay for gene expression employing an agent that substantially removes secondary structure of RNA.

In a fourth aspect, the present invention provides an assay for gene expression comprising:

(a) treating RNA with an agent that substantially removes secondary structure of the RNA;
(b) reverse transcribing and amplifying the RNA using primers capable of binding to complementary sequences of RNA; and
(c) measuring the presence or amount of treated and amplified RNA so as to obtain an indication of gene expression.

In a preferred form, the RNA is from an eukaryote or prokaryote including microorganism, cell, cells or a cell population.

In a preferred form, the RNA is mRNA.

In a preferred form, the RNA is from a microorganism.

The RNA can be obtained by any method suitable for isolating RNA from microorganisms, cells or cell population or other tissue or biological source. Such methods are well known in the art; see, for example, Sambrook et al, “Molecular Cloning, A Laboratory Manual” second ed., CSH Press, Cold Spring Harbor, 1989. Examples include but not limited to oligo-dT coated magnetic beads or resins. Specific examples of RNA binding resins specific examples include the following RNeasy™ and Oligotex™ (Qiagen), StrataPrep™ total (Stratagene), Nucleobond™ (Clontech), RNAgents™ and PolyATractT™ systems (Promega) etc. RNA may also be isolated using density gradient centrifugation techniques.

The RNA can be from eukaryotes or prokaryotes such as bacteria and viruses. The assay can be used for monitoring drug treatment, viral load, expression array for various viruses in a sample, and the like.

Preferably, the RNA is treated with an agent capable of modifying cytosine bases so as to weaken the binding strength between complementary regions of the RNA as removing the cytosines results in loss of C:G base pairing. The resulting modification removes secondary structure and substantially stabilizes the RNA as a single-stranded entity. The agent is preferably selected from bisulphite, hydroxylamine, acetate or citrate. More preferably, the agent is a bisulphite or acetate reagent. Most preferably, the agent is sodium bisulphite, a reagent which in the presence of water, modifies cytosine to uracil.

Sodium bisulphite (NaHSO₃) reacts readily with the 5,6-double bond of cytosine to form a sulfonated cytosine reaction intermediate which is susceptible to deamination, and in the presence of water gives rise to a uracil sulphite. If necessary, the sulphite group can be removed under mild alkaline conditions, resulting in the formation of uracil. Thus, potentially all cytosines will be converted to uracils. As uracil bases can form only two hydrogen bonds with any complementary base rather than the three hydrogen bonds which cytosines can form, the tendency for the RNA to reform complex secondary structures is greatly reduced. Thus treated, the modified RNA is then available to interact with specific complementary probes without encumbrance.

Importantly, in certain embodiments there is then no need to convert RNA to the corresponding complementary DNA (cDNA) as is the present practice before assaying the sample for its content of the target sequence. Because neither a reverse transcriptase nor a polymerase chain reaction (PCR) amplification step is required, the process according to the present invention is simpler, more direct, and hence less liable to error caused by a sequence copying bias of the enzymes involved in the standard procedures.

The amount of the target (modified) RNA present can be measured by any suitable means. For example, specific probes directed to the target RNA can be derived from part or all of the corresponding transcription unit of interest. Alternatively, the probes can be derived from any other entity which exhibits base-sequence specificity such an appropriate antibody or antibody fragment or single domain antibody, an oligonucleotide, or a peptide nucleic acid (PNA), locked nucleic acid (LNA) or intercalating nucleic acid (INA) probes of appropriate sequence.

The probes of the invention can be designed to be “substantially” complementary to the RNA to be tested. When the probes are PNA, LNA, oligonucleotide or INA in nature they would contain A (adenine), T (thymine), or C (cytosine) bases only because the modified RNA contains substantially no unmodified C residues.

The probes can be any suitable ligand such as oligonucleotide probes or PNA, LNA or INA probes. For example, a poly-T DNA or a poly-T PNA or an LNA probe or poly T INA probe can be used which will bind to total treated RNA, all of which have a poly A “tail”, from a cell and allow measurement of total gene expression in cells, cell population or tissue. Alternative, specific probes directed to an RNA of interest can be used to allow the measurement of specific gene expression in a given cell or tissue.

The replacement of cytosine with uracil, or its bisulphite adduct, in order to destabilise random secondary structure formation in the RNA also would significantly reduce the strength of binding of a specific oligo-, PNA, LNA, or INA probe with the modified RNA. An INA molecule when appropriately designed with an intercalating group restricted to terminal locations has enhanced binding characteristics to RNA of complementary sequence structure. To further compensate for this, in place of adenine bases in the probes it is preferred to substitute 2,6-diaminopurine (AP) which forms three hydrogen bonds with thymines (versus the two which adenine can form) in any complementary RNA strand and thus strengthen the binding between probe and RNA.

INA probes are constructed by attaching to various places in a sequence of ‘normal’ or ‘modified’ nucleotides an intercalating molecule which is capable of being inserted between adjoining bases of DNA or RNA exhibiting complementarity in its base sequence. With such DNA molecules, the presence of such intercalating moieties greatly stabilizes the interaction between probe and target nucleic acid no matter where the intercalating group is attached within the INA probe. The remarkable properties of INAs are described below.

In the case of INA probes designed to bind to RNA molecules, the intercalating groups are preferably placed at or close to the termini of the INA to enhance binding. Surprisingly, internal placement of intercalating groups may adversely affect hybridization of RNA to complementary DNA and can destabilize rather than stabilize the hybrid structure. Methods for constructing INA probes are described below.

The importance of the present invention relates to the surprising ability of INA probes of a particular construction to bind highly specifically and very tightly to target RNA species which has been treated to convert all its cytosine residues to uracil residues. As a consequence, the invention relates to methods which can avoid errors or biases introduced via the indirect processes of many of the methods presently in use.

Although, as indicated, a number of other specific probes can be used in this assay, it is preferred to use INA probes for reasons which will be apparent from the detailed description of their use.

Amplifying the RNA is preferably carried out using INA primers capable of binding to complementary sequences of RNA. The amplification would typically be carried out using reverse transcriptase PCR based methods.

With respect to equivalent sequences capable of hybridizing under high stringency conditions or having a high sequence similarity with nucleic acid molecules employed in the invention, “hybridizing under high stringency conditions” can be synonymous with “stringent hybridization conditions”, a term which is well known in the art; see, for example, Sambrook, “Molecular Cloning, A Laboratory Manual” second ed., CSH Press, Cold Spring Harbor, 1989; “Nucleic Acid Hybridisation, A Practical Approach”, Hames and Higgins eds., IRL Press, Oxford, 1985.

An advantage of the present invention is that direct measurement of RNA can be achieved without the need to convert RNA to cDNA. The assay allows a true measurement of gene activity in a cell population without introducing potential errors by the present methods that require conversion or amplification of RNA into cDNA.

PNA or oligonucleotide probes may be prepared using any suitable method known to the art. INA probes can be prepared by any suitable method known to the art.

It is also possible to amplify treated RNA from small amounts using INA primers prior to hybridization assays using suitable probes.

The present invention is suitable for use in current array technologies such as chips or in randomly addressable high density optical arrays so that large numbers of genes can be assayed rapidly. In this form, the activity of tens of thousands of genes can be measured or assayed in the one test. The invention is also adaptable to assays directed to small numbers of genes using bead technology, for example. Modified RNA species can be spotted or applied to suitable substrates in the form of an array and the array can be measured by various probes.

In one preferred form, the present invention makes particular use of the fact that PNA molecules have no net electrical charge while RNA molecules, because of their phosphate backbone, are highly negatively charged. Detection of bound PNA probes can utilize a simple molecule such as a positively charged fluorochrome, multiple molecules of which will bind specifically to nucleic acid in proportion to its length and can be directly detected. Many such suitable fluorochromes are known.

The detection system can also be an enzyme carrying a positively charged region that will selectively bind to the nucleic acid and that can be detected using an enzymatic assay, or a positively charged radioactive molecule that binds selectively to the captured nucleic acid. It will be appreciated that nanocrystals could also be used.

Another suitable detection system is the use of quantum dot bioconjugates (Chan and Nie 1998 Science 282: 2016-2018).

Alternatively, microspheres, to which are attached sequence specific probes together with a number of fluorochrome molecules, can be utilized. The microspheres can be attached directly to the probes targeting a particular RNA species, or via secondary non-specific component part of the RNA such as its polyadenine tail. In this latter instance, the attachment of the microsphere signal detection system could be via a poly T sequence as an INA, PNA, LNA or oligonucleotide entity.

As microspheres carrying fluorochrome markers come in a variety of colours or spectra, it is possible in a single experiment to measure the amount of each of several different RNA species present in a single cell sample. Moreover, single microspheres, so labelled, can be readily visualised and counted, so small differences in expression between different RNA species can be determined with considerable accuracy.

Other methods for detecting ligands binding to target modified RNA, such as labelling with a suitable radioactive compound or an enzyme capable of reacting with a substrate to formed a colored product, could also be used for particular applications either attached directly to the capture RNA or the probe or the substrate.

Using INA or PNA or other oligonucleotide probes as one of the ligands in this procedure has very significant advantages over the use of oligonucleotide probes. INA or PNA binding reaches equilibrium faster and exhibits greater sequence specificity. PNA molecules are uncharged and can bind the target modified RNA molecules with a higher binding coefficient than conventional oligonucleotide probes. In particular, INA probes enhance binding between A- T- and A-U bases. This is of importance in the instance of RNA which has been treated to remove secondary structure in which cytosine bases are converted to uracil bases. As a consequence of RNA treatment, there are fewer G-C base interactions and a corresponding increase in the number of A-T plus A-U base interactions.

As the invention can use direct detection methods, the assay can provide a true and accurate measure of the amount of a target RNA in a sample. The assay is not confounded by potential bias inherent in methods that rely for signal amplification on processes such as PCR, where the enzymes commonly used in such procedures can introduce systematic bias through differential rates of amplification of different sequences.

The present invention is particularly suitable for detection of disease states, differentiation states of stem cells and derivative cell populations, detection or measurement of effects of medication on gene expression or cellular function, and any other situation where an accurate indication of gene expression is useful such as viral load monitoring to assist in the determination of the correct drug regime for patients infected with viruses such as Hepatitis C virus (HCV) and human immunodeficiency virus (HIV).

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of the invention.

In order that the present invention may be more clearly understood, preferred forms will be described with reference to the following drawings and examples.

### BRIEF DESCRIPTION OF THE DRAWINGS

FIG. 1A shows a representation of a typical micro-array based assay where dark spots indicate genes that are up-regulated in a certain RNA population and light spots indicate down-regulated genes within the same population. Dark and light arrows indicate genes that are detected as being up-regulated and down-regulated respectively in the bisulphite treated RNA but not in conventional methods due to secondary structure which prevents the detector molecules in the conventional system from binding to their target.

FIG. 1B shows a micro-array based assay similar to FIG. 1A but dark and light arrows indicate genes that are detected as being up-regulated and down-regulated respectively in the bisulphite treated RNA but not in conventional methods due to bias produced during enzymatic manipulation of the RNA prior to expression analysis, resulting in incorrect determination of RNA expression levels. Enzymatic manipulations can cause misleading results and indicate that certain genes are up or down regulated when in fact they are not so regulated.

FIG. 1C shows a micro-array based assay similar to FIG. 1A but dark and light arrows indicate genes that are detected as being up-regulated and down-regulated respectively in the bisulphite treated RNA but not in conventional methods due to improved specificity of the detector molecules. Increasing the specific binding strength of the detector molecules leads to the detection of RNA species which may not be detected using conventional methods due to lack of specificity.

FIG. 2 shows gel separation results of PCR Amplification from bisulphite treated RNA. Separation wells: M, 100-1000 by marker; Lane 5 Wild type Actin primers exon 3a-3b; Lane 6 Wild type Actin primers exon 3a-4; Lane 7 bisulphite converted Actin primers exon 3a-3b; Lane 8 Wild type Actin primers exon 3a-4.

FIG. 3 shows sequence analysis of Actin RNA. A. bisulphite treated converted Actin RNA (SEQ ID NO: 1) and B. wild type Actin RNA (SEQ ID NO: 2).

FIG. 4 shows linearity performance gel based readout for Hepatitis C virus (HCV).

FIG. 5 shows real time PCR Quantitation Report—Linearity panel for Hepatitis C virus (HCV) RNA.

FIG. 6 shows real time PCR Quantitation Report—Dynamic range for Hepatitis C virus (HCV).

### MODE(S) FOR CARRYING OUT THE INVENTION

### Definitions

### Nucleic Acids

The term “nucleic acid” covers the naturally occurring nucleic acids, DNA and RNA. The term “nucleic acid analogues” covers derivatives of the naturally occurring nucleic acids, DNA and RNA, as well as synthetic analogues of naturally occurring nucleic acids. Synthetic analogues comprise one or more nucleotide analogues. The term nucleotide analogue includes all nucleotide analogues capable of being incorporated into a nucleic acid backbone and capable of specific base-pairing (see below), essentially like naturally occurring nucleotides.

Hence the terms “nucleic acid” or “nucleic acid analogues” designate any molecule which essentially consists of a plurality of nucleotides and/or nucleotide analogues and/or intercalator pseudonucleotides. Nucleic acids or nucleic acid analogues useful for the present invention may comprise a number of different nucleotides with different backbone monomer units.

Preferably, single strands of nucleic acids or nucleic acid analogues are capable of hybridising with a substantially complementary single stranded nucleic acid and/or nucleic acid analogue to form a double stranded nucleic acid or nucleic acid analogue. More preferably such a double stranded analogue is capable of forming a double helix. Preferably, the double helix is formed due to hydrogen bonding, more preferably, the double helix is a double helix selected from the group consisting of double helices of A form, B form, Z form and intermediates thereof.

Hence, nucleic acids and nucleic acid analogues useful for the present invention include, but is not limited to DNA, RNA, LNA, PNA, MNA, ANA, HNA and mixtures thereof and hybrids thereof, as well as phosphorous atom modifications thereof, such as but not limited to phosphorothioates, methyl phospholates, phosphoramidites, phosphorodithiates, phosphoroselenoates, phosphotriesters and phosphoboranoates. In addition non-phosphorous containing compounds may be used for linking to nucleotides such as but not limited to methyliminomethyl, formacetate, thioformacetate and linking groups comprising amides. In particular nucleic acids and nucleic acid analogues may comprise one or more intercalator pseudonucleotides.

Within this context “mixture” is meant to cover a nucleic acid or nucleic acid analogue strand comprising different kinds of nucleotides or nucleotide analogues. Furthermore, within this context, “hybrid” is meant to cover nucleic acids or nucleic acid analogues comprising one strand which comprises nucleotide or nucleotide analogue with one or more kinds of backbone and another strands which comprises nucleotide or nucleotide analogue with different kinds of backbone.

By HNA is meant nucleic acids as for example described by Van Aetschot et al., 1995. By MNA is meant nucleic acids as described by Hossain et al, 1998. ANA refers to nucleic acids described by Allert et al, 1999. LNA may be any LNA molecule as described in WO 99/14226 (Exiqon), preferably, LNA is selected from the molecules depicted in the abstract of WO 99/14226. More preferably, LNA is a nucleic acid as described in Singh et al, 1998, Koshkin et al, 1998 or Obika et al., 1997. PNA refers to peptide nucleic acids as for example described by Nielsen et al, 1991.

The term nucleotide designates the building blocks of nucleic acids or nucleic acid analogues and the term nucleotide covers naturally occurring nucleotides and derivatives thereof as well as nucleotides capable of performing essentially the same functions as naturally occurring nucleotides and derivatives thereof. Naturally occurring nucleotides comprise deoxyribonucleotides comprising one of the four main nucleobases adenine (A), thymine (T), guanine (G) or cytosine (C), and ribonucleotides comprising on of the four nucleobases adenine (A), uracil (U), guanine (G) or cytosine (C).

Nucleotide analogues may be any nucleotide like molecule that is capable of being incorporated into a nucleic acid backbone and capable of specific base-pairing.

Non-naturally occurring nucleotides includes, but is not limited to the nucleotides comprised within DNA, RNA, PNA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R-RNA, α-L-RNA or α-D-RNA, 8-D-RNA.

The function of nucleotides and nucleotide analogues is to be able to interact specifically with complementary nucleotides via hydrogen bonding of the nucleobases of the complementary nucleotides as well as to be able to be incorporated into a nucleic acid or nucleic acid analogue. Naturally occurring nucleotide, as well as some nucleotide analogues are capable of being enzymatically incorporated into a nucleic acid or nucleic acid analogue, for example by RNA or DNA polymerases. However, nucleotides or nucleotide analogues may also be chemically incorporated into a nucleic acid or nucleic acid analogue.

Furthermore nucleic acids or nucleic acid analogues may be prepared by coupling two smaller nucleic acids or nucleic acid analogues to another, for example this may be done enzymatically by ligases or it may be done chemically.

Nucleotides or nucleotide analogues comprise a backbone monomer unit and a nucleobase. The nucleobase may be a naturally occurring nucleobase or a derivative thereof or an analogue thereof capable of performing essentially the same function. The function of a nucleobase is to be capable of associating specifically with one or more other nucleobases via hydrogen bonds. Thus it is an important feature of a nucleobase that it can only form stable hydrogen bonds with one or a few other nucleobases, but that it can not form stable hydrogen bonds with most other nucleobases usually including itself. The specific interaction of one nucleobase with another nucleobase is generally termed “base-pairing”.

The base pairing results in a specific hybridisation between predetermined and complementary nucleotides. Complementary nucleotides are nucleotides that comprise nucleobases that are capable of base-pairing.

Of the common naturally occurring nucleobases, adenine (A) pairs with thymine (T) or uracil (U); and guanine (G) pairs with cytosine (C). Accordingly, a nucleotide comprising A is complementary to a nucleotide comprising either T or U, and a nucleotide comprising G is complementary to a nucleotide comprising C.

Nucleotides may further be derivatised to comprise an appended molecular entity. The nucleotides can be derivatised on the nucleobases or on the backbone monomer unit. Preferred sites of derivatisation on the bases include the 8-position of adenine, the 5-position of uracil, the 5- or 6-position of cytosine, and the 7-position of guanine. The heterocyclic modifications can be grouped into three structural classes: Enhanced base stacking, additional hydrogen bonding, and the combination of these classes. Modifications that enhance base stacking by expanding the π-electron cloud of the planar systems are represented by conjugated, lipophilic modifications in the 5-position of pyrimidines and the 7-position of 7-deaza-purines. Substitutions in the 5-position of pyrimidines modifications include propynes, hexynes, thiazoles and simply a methyl group; and substituents in the 7-position of 7-deaza purines include iodo, propynyl, and cyano groups. It is also possible to modify the 5-position of cytosine from propynes to five-membered heterocycles and to tricyclic fused systems, which emanate from the 4- and 5-position (cytosine clamps). A second type of heterocycle modification is represented by the 2-amino-adenine where the additional amino group provides another hydrogen bond in the A-T base pair, analogous to the three hydrogen bonds in a G-C base pair. Heterocycle modifications providing a combination of effects are represented by 2-amino-7-deaza-7-modified adenine and the tricyclic cytosine analog having an ethoxyamino functional group of heteroduplexes. Furthermore, N2-modified 2-amino adenine modified oligonucleotides are among commonly modifications. Preferred sites of derivatisation on ribose or deoxyribose moieties are modifications of non-connecting carbon positions C-2′ and C-4′, modifications of connecting carbons C-1′, C-3′ and C-5′, replacement of sugar oxygen, O-4′, anhydro sugar modifications (conformational restricted), cyclosugar modifications (conformational restricted), ribofuranosyl ring size change, connection sites—sugar to sugar, (C-3′ to C-5′/C-2′ to C-5′), hetero-atom ring—modified sugars and combinations of above modifications. However, other sites may be derivatised, as long as the overall base pairing specificity of a nucleic acid or nucleic acid analogue is not disrupted. Finally, when the backbone monomer unit comprises a phosphate group, the phosphates of some backbone monomer units may be derivatised.

Oligonucleotide or oligonucleotide analogue as used herein are molecules essentially consisting of a sequence of nucleotides and/or nucleotide analogues and/or intercalator pseudonucleotides. Preferably oligonucleotide or oligonucleotide analogue comprises 5 to 100 individual nucleotides. Oligonucleotide or oligonucleotide analogues may comprise DNA, RNA, LNA, 2′-O-methyl RNA, PNA, ANA, HNA and mixtures thereof, as well as any other nucleotide and/or nucleotide analogue and/or intercalator pseudonucleotide.

### RNA

As used herein, RNA includes messenger RNA (mRNA) immature mRNA, transfer RNA (tRNA), ribosomal RNA (rRNA) and microRNA (miRNA) from any source such as cells, genomic RNA from viruses or other microorganisms, transcribed RNA from DNA, RNA copy of corresponding DNA, and the like.

### Corresponding Nucleic Acids

Nucleic acids, nucleic acid analogues, oligonucleotides or oligonucleotides analogues are considered to be corresponding when they are capable of hybridising. Preferably corresponding nucleic acids, nucleic acid analogues, oligonucleotides or oligonucleotides analogues are capable of hybridising under low stringency conditions, more preferably corresponding nucleic acids, nucleic acid analogues, oligonucleotides or oligonucleotides analogues are capable of hybridising under medium stringency conditions, more preferably corresponding nucleic acids, nucleic acid analogues, oligonucleotides or oligonucleotides analogues are capable of hybridising under high stringency conditions.

High stringency conditions as used herein shall denote stringency as normally applied in connection with Southern blotting and hybridisation as described e.g. by Southern E. M., 1975, J. Mol. Biol. 98:503-517. For such purposes it is routine practise to include steps of prehybridization and hybridization. Such steps are normally performed using solutions containing 6×SSPE, 5% Denhardt's, 0.5% SDS, 50% formamide, 100 μg/ml denatured salmon testis DNA (incubation for 18 hrs at 42° C.), followed by washing with 2×SSC and 0.5% SDS (at room temperature and at 37° C.), and washing with 0.1×SSC and 0.5% SDS (incubation at 68° C. for 30 min), as described by Sambrook et al., 1989, in “Molecular Cloning/A Laboratory Manual”, Cold Spring Harbor).

Medium stringency conditions as used herein shall denote hybridisation in a buffer containing 1 mM EDTA, 10 mM Na₂HPO₄·H₂0, 140 mM NaCl, at pH 7.0. Preferably, around 1.5 μM of each nucleic acid or nucleic acid analogue strand is provided. Alternatively medium stringency may denote hybridisation in a buffer containing 50 mM KCl, 10 mM TRIS-HCl (pH 9,0), 0.1% Triton X-100, 2 mM MgCl₂.

Low stringency conditions denote hybridisation in a buffer constituting 1 M NaCl, 10 mM Na₃PO₄ at pH 7.0.

Alternatively, corresponding nucleic acids, nucleic acid analogues, oligonucleotides or oligonucleotides, nucleic acid analogues, oligonucleotides or oligonucleotides substantially complementary to each other over a given sequence, such as more than 70% complementary, for example more than 75% complementary, such as more than 80% complementary, for example more than 85% complementary, such as more than 90% complementary, for example more than 92% complementary, such as more than 94% complementary, for example more than 95% complementary, such as more than 96% complementary, for example more than 97% complementary.

Preferably the given sequence is at least 10 nucleotides long, such as at least 15 nucleotides, for example at least 20 nucleotides, such as at least 25 nucleotides, for example at least 30 nucleotides, such as between 10 and 500 nucleotides, for example between 10 and 100 nucleotides long, such as between 10 and 50 nucleotides long. More preferably corresponding oligonucleotides or oligonucleotides analogues are substantially complementary over their entire length.

### Cross-Hybridisation

The term cross-hybridisation covers unintended hybridisation between at least two nucleic acids or nucleic acid analogues. Hence the term cross-hybridization may be used to describe the hybridisation of for example a nucleic acid probe or nucleic acid analogue probe sequence to other nucleic acid sequences or nucleic acid analogue sequences than its intended target sequence.

Often cross-hybridization occurs between a probe and one or more corresponding non-target sequences, even though these have a lower degree of complementarity than the probe and its corresponding target sequence. This unwanted effect could be due to a large excess of probe over target and/or fast annealing kinetics. Cross-hybridization also occurs by hydrogen bonding between few nucleobase pairs, e.g. between primers in a PCR reaction, resulting in primer dimer formation and/or formation of unspecific PCR products.

Nucleic acids comprising one or more nucleotide analogues with high affinity for nucleotide analogues of the same type tend to form dimer or higher order complexes based on base pairing. Probes comprising nucleotide analogues such as, but not limited to, LNA, 2′-O-methyl RNA and PNA generally have a high affinity for hybridising to other oligonucleotide analogues comprising backbone monomer units of the same type. Hence even though individual probe molecules only have a low degree of complementarity they tend to hybridize.

### Self-Hybridisation

The term self-hybridisation covers the process wherein a nucleic acid or nucleic acid analogue molecule anneals to itself by folding back on itself, generating a secondary structure like for example a hairpin structure. In most applications it is of importance to avoid self-hybridization. The generation of secondary structures may inhibit hybridisation with desired nucleic acid target sequences. This is undesired in most assays for example when the nucleic acid or nucleic acid analogue is used as primer in PCR reactions or as fluorophore/quencher labelled probe for exonuclease assays. In both assays, self-hybridisation will inhibit hybridization to the target nucleic acid and additionally the degree of fluorophore quenching in the exonuclease assay is lowered.

Nucleic acids comprising one or more nucleotide analogues with high affinity for nucleotide analogues of the same type tend to self-hybridize. Probes comprising nucleotide analogues such as, but not limited to, LNA, 2′-O-methyl RNA and PNA generally have a high affinity for self-hybridising. Hence even though individual probe molecules only have a low degree of self-complementary they tend to self-hybridize.

### Melting Temperature

Melting of nucleic acids refer to the separation of the two strands of a double-stranded nucleic acid molecule. The melting temperature (Tₘ) denotes the temperature in degrees celsius at which 50% helical (hybridized) versus coil (unhybridized) forms are present.

A high melting temperature is indicative of a stable complex and accordingly of a high affinity between the individual strands. Similarly, a low melting temperature is indicative of a relatively low affinity between the individual strands. Accordingly, usually strong hydrogen bonding between the two strands results in a high melting temperature.

Furthermore, intercalation of an intercalator between nucleobases of a double stranded nucleic acid may also stabilise double stranded nucleic acids and accordingly result in a higher melting temperature.

In addition, the melting temperature is dependent on the physical/chemical state of the surroundings. For example the melting temperature is dependent on salt concentration and pH.

The melting temperature may be determined by a number of assays, for example it may be determined by using the UV spectrum to determine the formation and breakdown (melting) of hybridisation.

### INA/IPN Definition

Intercalating Nucleic Acids (INAs) are a unique class of DNA binding molecules. INAs are comprised of nucleotides and/or nucleotide analogues and intercalating pseudonucleotide (IPN) monomers. INAs have a very high affinity for complementary DNA with stabilisations of up to 10 degrees for internally placed IPNs and up to 11 degrees for end position IPNs. The INA itself if designed correctly can be a selective molecule that prefers to hybridise with DNA over complementary RNA. It has been shown that INAs bind about 25 times less efficiently to RNA than oligonucleotide primers if the IPN's are placed internally in the molecule. Whereas, conventional oligonucleotides, oligonucleotide analogues and PNAs have an equal affinity for both RNA and DNA. Thus INAs are the first truly selective DNA binding agents. In addition, INAs have a higher specificity and affinity for complementary DNA that other natural DNA molecules.

In addition, IPNs stabilise DNA best in AT-rich surroundings which make them especially useful in the field of epigenomics research. The IPNs are typically placed as bulge or end insertions in to the INA molecule. The IPN is essentially a planar (hetero) polyaromatic compound that is capable of co-stacking with nucleobases in a nucleic acid duplex.

The INA molecule has also been shown to be resistant to exonuclease attack. This makes these molecules especially useful as primers for amplification using enzymes such as phi29. As phi29 has inherent exonuclease activity, primers used as templates for amplification must be specially modified at their 3′ terminus to prevent enzyme degradation. INA molecules, however, can be added without further modification.

INAs can be used in conventional PCR amplification reactions and behave as conventional primers. INAs, however, have a higher specificity for DNA or RNA templates making them ideal for the use in situations where template is limiting and sensitivity of the reaction is critical. INAs stabilise DNA best in AT-rich surroundings which make them especially useful for amplification of bisulphite treated DNA sequences. This is due to the fact that after bisulphite conversion, all the cytosine residues are converted to uracil and subsequently thymine after PCR or other amplification. Bisulphite treated DNA is therefore very T rich. Increasing the number of IPN molecules in the INA results in increased stabilization of the INA/DNA duplex. The more IPNs in the INA, the greater the melting temperature of the DNA/INA duplex.

The present applicant has previously developed a class of intercalator pseudonucleotides which, when incorporated into an oligonuceotide or oligonuceotide analogue, form an intercalating nucleic acid (INA) (WO 03/051901, WO 03/052132, WO 03/052133 and WO 03/052134) which has novel and useful properties as a supplement to, or replacement of, oligonucleotides.

The intercalator pseudonucleotide is preferably selected from phosphoramidites of 1-(4,4′-dimethoxytriphenylmethyloxy)-3-pyrenemethyloxy-2-propanol. Preferably, the intercalator pseudonucleotide is selected from the phosphoramidite of (S)-1-(4,4′-dimethoxytriphenylmethyloxy)-3-pyrenemethyloxy-2-propanol or the phosphoramidite of (R)-1-(4,4′-dimethoxytriphenylmethyloxy)-3-pyrenemethyloxy-2-propanol.

The oligonucleotide or oligonucleotide analogue can be selected from DNA, RNA, locked nucleic acid (LNA), peptide nucleic acid (PNA), MNA, altritol nucleic acid (ANA), hexitol nucleic acid (HNA), intercalating nucleic acid (INA), cyclohexanyl nucleic acid (CNA) and mixtures thereof and hybrids thereof, as well as phosphorous atom modifications thereof, such as but not limited to phosphorothioates, methyl phospholates, phosphoramidites, phosphorodithiates, phosphoroselenoates, phosphotriesters and phosphoboranoates. Non-naturally occurring nucleotides include, but not limited to the nucleotides comprised within DNA, RNA, PNA, INA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R-RNA, α-L-RNA or α-D-RNA, β-D-RNA. In addition non-phosphorous containing compounds may be used for linking to nucleotides such as but not limited to methyliminomethyl, formacetate, thioformacetate and linking groups comprising amides. In particular nucleic acids and nucleic acid analogues may comprise one or more intercalator pseudonucleotides.

When IPNs are placed in an INA molecule for the specific detection of methylated sites, the present inventor has found that it is useful to avoid placing an IPN between potential CpG sites. This is due to the fact that when a CpG site is split using an IPN the specificity of the resulting INA is reduced.

### Peptide Nucleic Acid (PNA)

Peptide nucleic acids are non-naturally occurring polyamides which can hybridize to nucleic acids (DNA and RNA) with sequence specificity. (See U.S. Pat. No. 5,539,082 and Egholm et al., Nature (1993) 365, 566-568). PNAs are candidates as alternatives/substitutes to nucleic acid probes in probe-based hybridization assays because they exhibit several desirable properties. PNAs are achiral polymers which hybridize to nucleic acids to form hybrids which are more thermodynamically stable than a corresponding nucleic acid/nucleic acid complex. Being non-naturally occurring molecules, they are not known to be substrates for the enzymes which are known to degrade peptides or nucleic acids. Therefore, PNAs should be stable in biological samples, as well as, have a long shelf-life. Unlike nucleic acid hybridization which is very dependent on ionic strength, the hybridization of a PNA with a nucleic acid is fairly independent of ionic strength and is favoured at low ionic strength under conditions which strongly disfavour the hybridization of nucleic acid to nucleic acid. The effect of ionic strength on the stability and conformation of PNA complexes has been extensively investigated. Sequence discrimination is more efficient for PNA recognizing DNA or RNA than for DNA recognizing DNA. However, the advantages in single base change, indel, or polymorphism discrimination with PNA probes, as compared with DNA probes, in a hybridization assay appears to be somewhat sequence dependent. As an additional advantage, PNAs hybridize to nucleic acid in both a parallel and antiparallel orientation, though the antiparallel orientation is preferred.

PNAs are synthesized by adaptation of standard peptide synthesis procedures in a format which is now commercially available. (For a general review of the preparation of PNA monomers and oligomers please see: Dueholm et al., New J. Chem. (1997), 21, 19-31 or Hyrup et. al., Bioorganic & Med. Chem. (1996) 4, 5-23). Labelled and unlabelled PNA oligomers can be purchased (See: PerSeptive Biosystems Promotional Literature: BioConcepts, Publication No. NL612, Practical PNA, Review and Practical PNA, Vol. 1, Iss. 2) or prepared using the commercially available products.

There are indeed many differences between PNA probes and standard nucleic acid probes. These differences can be conveniently broken down into biological, structural, and physico-chemical differences. As discussed above and below, these biological, structural, and physico-chemical differences may lead to unpredictable results when attempting to use PNA probes in applications were nucleic acids have typically been employed. This non-equivalency of differing compositions is often observed in the chemical arts.

With regard to biological differences, nucleic acids are biological materials that play a central role in the life of living species as agents of genetic transmission and expression. Their in vivo properties are fairly well understood. PNA, however, is a recently developed totally artificial molecule, conceived in the minds of chemists and made using synthetic organic chemistry. It has no known biological function.

Structurally, PNA also differs dramatically from nucleic acid. Although both can employ common nucleobases (A, C, G, T, and U), the backbones of these molecules are structurally diverse. The backbones of RNA and DNA are composed of repeating phosphodiester ribose and 2-deoxyribose units. In contrast, the backbones of PNA are composed on N-(2-aminoethyl)glycine units. Additionally, in PNA the nucleobases are connected to the backbone by an additional methylene carbonyl unit.

Despite its name, PNA is not an acid and contains no charged acidic groups such as those present in DNA and RNA. Because they lack formal charge, PNAs are generally more hydrophobic than their equivalent nucleic acid molecules. The hydrophobic character of PNA allows for the possibility of non-specific (hydrophobic/hydrophobic interactions) interactions not observed with nucleic acids. Furthermore, PNA is achiral, providing it with the capability of adopting structural conformations the equivalent of which do not exist in the RNA/DNA realm.

The physico/chemical differences between PNA and DNA or RNA are also substantial. PNA binds to its complementary nucleic acid more rapidly than nucleic acid probes bind to the same target sequence. This behaviour is believed to be, at least partially, due to the fact that PNA lacks charge on its backbone. Additionally, recent publications demonstrate that the incorporation of positively charged groups into PNAs will improve the kinetics of hybridization. Because it lacks charge on the backbone, the stability of the PNA/nucleic acid complex is higher than that of an analogous DNA/DNA or RNA/DNA complex. In certain situations, PNA will form highly stable triple helical complexes or form small loops through a process called “strand displacement”. No equivalent strand displacement processes or structures are known in the DNA/RNA world.

In summary, because PNAs hybridize to nucleic acids with sequence specificity, PNAs are useful candidates for developing probe-based assays. Importantly, PNA probes are not the equivalent of nucleic acid probes. Nonetheless, even under the most stringent conditions both the exact target sequence and a closely related sequence (e.g. a non-target sequence having a single point mutation (single base pair mismatch)) will often exhibit detectable interaction with a labelled nucleic acid or labelled PNA probe. Any hybridization to a closely related non-target sequence will result in the generation of undesired background signal. Because the sequences are so closely related, point mutations are some of the most difficult of all nucleic acid modifications to detect using a probe-based assay. Numerous diseases, such as sickle cell anemia and cystic fibrosis, are sometimes caused by a single point mutation of genomic nucleic acid. Consequently, any method, kits or compositions which could improve the specificity, sensitivity and reliability of probe-based assays would be useful in the detection, analysis and quantitation of DNA containing samples.

### Sodium Bisulphite

Methods for treating nucleic acid with sodium bisuphite can be found in a number of references including Frommer et al 1992, Proc Natl Acad Sci 89:1827-1831; Grigg and Clark 1994 BioAssays 16:431-436; Shapiro et al 1970, J Amer Chem Soc 92:422 to 423; Wataya and Hayatsu 1972, Biochemistry 11:3583-3588.

Methods have also been developed by the present applicant to improve or enhance success of bisulphite treatment of nucleic acids.

An exemplary protocol for effective bisulphite treatment of nucleic acid is set out below. The protocol results in retaining substantially all DNA treated. This method is also referred to herein as the Human Genetic Signatures (HGS) method. It will be appreciated that the volumes or amounts of sample or reagents can be varied.

Preferred method for bisulphite treatment can be found in U.S. Ser. No. 10/428,310 or PCT/AU2004/000549.

To 2 μg of DNA, which can be pre-digested with suitable restriction enzymes if so desired, 2 μl ( 1/10 volume) of 3 M NaOH (6 g in 50 ml water, freshly made) was added in a final volume of 20 μl. This step denatures the double stranded DNA molecules into a single stranded form, since the bisulphite reagent preferably reacts with single stranded molecules. The mixture was incubated at 37° C. for 15 minutes. Incubation at temperatures above room temperature can be used to improve the efficiency of denaturation.

After the incubation, 208 μl 2 M Sodium Metabisulphite (7.6 g in 20 ml water with 416 ml 10 N NaOH; BDH AnalaR #10356.40; freshly made) and 12 μl of 10 mM Quinol (0.055 g in 50 ml water, BDH AnalR #103122E; freshly made) were added in succession. Quinol is a reducing agent and helps to reduce oxidation of the reagents. Other reducing agents can also be used, for example, dithiothreitol (DTT), mercaptoethanol, quinone (hydroquinone), or other suitable reducing agents. The sample was overlaid with 200 μl of mineral oil. The overlaying of mineral oil prevents evaporation and oxidation of the reagents but is not essential. The sample was then incubated overnight at 55° C. Alternatively the samples can be cycled in a thermal cycler as follows: incubate for about 4 hours or overnight as follows: Step 1, 55° C./2 hr cycled in PCR machine; Step 2, 95° C./2 min. Step 1 can be performed at any temperature from about 37° C. to about 90° C. and can vary in length from 5 minutes to 8 hours. Step 2 can be performed at any temperature from about 70° C. to about 99° C. and can vary in length from about 1 second to 60 minutes, or longer.

After the treatment with Sodium Metabisulphite, the oil was removed, and 1 μl tRNA (20 mg/ml) or 2 μl glycogen were added if the DNA concentration was low. These additives are optional and can be used to improve the yield of DNA obtained by co-precitpitating with the target DNA especially when the DNA is present at low concentrations. The use of additives as carrier for more efficient precipitation of nucleic acids is generally desired when the amount nucleic acid is <0.5 μg.

An isopropanol cleanup treatment was performed as follows: 800 μl of water were added to the sample, mixed and then 1 ml isopropanol was added. The water or buffer reduces the concentration of the bisulphite salt in the reaction vessel to a level at which the salt will not precipitate along with the target nucleic acid of interest. The dilution is generally about ¼ to 1/1000 so long as the salt concentration is diluted below a desired range, as disclosed herein.

The sample was mixed again and left at 4° C. for a minimum of 5 minutes. The sample was spun in a microfuge for 10-15 minutes and the pellet was washed 2× with 70% ETOH, vortexing each time. This washing treatment removes any residual salts that precipitated with the nucleic acids.

The pellet was allowed to dry and then resuspended in a suitable volume of T/E (10 mM Tris/0.1 mM EDTA) pH 7.0-12.5 such as 50 μl. Buffer at pH 10.5 has been found to be particularly effective. The sample was incubated at 37° C. to 95° C. for 1 min to 96 hr, as needed to suspend the nucleic acids.

Agent that Substantially Removes Secondary Structure of RNA

Agents suitable for the present invention include bisulphite, hydroxylamine, acetate or citrate. Bisulphite reagents are preferred and include sodium bisulphite, sodium metabisulphite, and guanidinium hydrogen sulphite as described in WO 2005054502. In this regard, the treatment RNA can be carried out wherein guanidinium hydrogen sulphite is used for the preparation of a solution containing guanidinium ions and sulphite ions and subsequent treatment of the RNA.

### Cell Lines

TABLE 1
Cell lines used for RNA analysis

| NAME | CELL TYPE | GROWING CONDITIONS |
|---|---|---|
| HeLa | Cervical | RPM1 + 10% HI FCS for initial rapid |
| | Carcinoma | growth then DMEM + 10% HI FCS for |
| | | slower growth. Split 1:10, 2x week |
| HepG2 | Liver | DMEM (high glucose 4.5 g/L) + 10% HI |
| | Carcinoma | FCS + 2 mM Glutamine. |
| | | Split 1:4 2x week |

RNA Extraction from Cells
I. 1 ml of Trizol was added directly to the cells (90% confluent) after removal of media.
II. Samples mixed well and left at room temperature for 5 minutes to dissociate nucleoprotein complexes.
III. 0.5 ml removed into a clean RNase free 1.5 ml centrifuge tube.
IV. The samples were then spun @ 12,000×g for 10 minutes@ 4° C. to remove high molecular weight DNA and other contaminants.
V. The supernatant removed into a clean tube and 100 μl of 100% chloroform added and the samples mixed vigorously by hand for 15 seconds then incubated at room temperature for 2-3 minutes.
VI. The samples were then spun @ 12,000×g for 10 minutes at 4° C. to separate the phases.
VII. The upper aqueous phase was removed into a clean tube ensuring the pipette tip stayed away from the interface and 1 μl of 20 mg/ml glycogen added and the samples vortexed.
VIII. An equal volume of 100% isopropanol (0.25 ml) was added the tubes vortexed then left at room temp for 10 minutes.
IX. The samples were then spun @ 12,000×g for 10 minutes at 4° C. to pellet the RNA.
X. The supernatant removed and the pellet washed with 0.75 ml of 80% ethanol to removed inhibitors of the cDNA synthesis reaction, vortexed briefly then spun @ 7,500×g for 5 minutes at 4° C. to pellet the RNA.
XI. Step X was repeated a further time.
XII. The pellet was then spun in a microfuge for 10 seconds the residual ethanol removed and the pellet immediately resuspended in 25 μl of RNase free water. NB if the pellet dries out then it is very difficult to resuspend the RNA and the 260/280 ratio will be less than 1.6.
XIII. The OD _{260/280/310} was then recorded and the RNA stored at ±70° C. until required.

### Preparation of RNA

The RNA sample was resuspended in 20 μl of nuclease free water after extraction from the desired cells or tissue.

The sample was heated at 60-100° C. for 2-3 minutes to resolve secondary structure and immediately used in the bisulphite reaction.

### Bisulphite Treatment

An exemplary protocol demonstrating the effectiveness of the bisulphite treatment of RNA according to the present invention is set out below. The protocol successfully resulted in retaining substantially all RNA treated. This method of the invention is also referred to herein as the Human Genetic Signatures (HGS) method. It will be appreciated that the volumes or amounts of sample or reagents can be varied.

2 μg of RNA is resuspended in a total of 20 μl RNase free water. The sample was then incubated at 65° C. for 2 minutes to remove secondary structure. After the incubation, 208 μl 2 M Sodium Metabisulphite pH 5.0 (7.6 g in 20 ml water or 10 mM Tris/1 mM EDTA with 416 ml 10 N NaOH; BDH AnalaR #10356.4D; freshly made) was added in succession. RNase inhibitors can also be added at this point such as RNaseOUT (invitrogen cat #10777-019) according to the manufacturers instructions. The sample was overlaid with 200 μl of mineral oil. The overlaying of mineral oil prevents evaporation and oxidation of the reagents but is not essential. The sample was then incubated overnight at 55° C. This incubation can be performed at any temperature from about 37° C. to about 90° C. and can vary, in length from 5 minutes to 16 hours.

After the treatment with Sodium Metabisulphite, the oil was removed, and 1 μl glycogen (20 mg/ml) was added especially if the RNA concentration was low. This additive is optional and can be used to improve the yield of RNA obtained by co-precitpitating with the target RNA especially when the RNA is present at low concentrations. The use of additives as carrier for more efficient precipitation of nucleic acids is generally desired when the amount nucleic acid is <0.5 μg.

An isopropanol cleanup treatment was performed as follows: 800 μl of RNase free water was added to the sample, mixed and then 1 ml isopropanol was added. The water or buffer reduces the concentration of the bisulphite salt in the reaction vessel to a level at which the salt will not precipitate along with the target nucleic acid of interest. The dilution is generally about ¼ to 1/1000 so long as the salt concentration is diluted below a desired range, as disclosed herein.

The sample was mixed again and left at 4° C. for a minimum of 5 minutes but can be up to 60 minutes. The sample was spun in a microfuge for 10-15 minutes and the pellet was washed 2× with 80% ETOH. This washing treatment removes any residual salts that precipitated with the nucleic acids.

The pellet was allowed to dry briefly to remove residual ethanol but ensuring that the pellet did not dry out totally as this can reduce the final RNA yield and then resuspended in a suitable volume of T/E (10 mM Tris/0.1 mM EDTA) pH 7.0-12.5 such as 50 μl. RNase inhibitors can also be added at this point such as RNaseOUT (invitrogen cat #10777-019) according to the manufacturers instructions. Buffer at pH 10.5 has been found to be particularly effective. The sample was incubated at 37° C. to 95° C. for 1 min to 96 hr, as needed to suspend the nucleic acids.

cDNA Synthesis

The following reagents were prepared for each cDNA synthesis reaction in thin-wall 0.5 ml RNase free tubes.

| | | |
|---|---|---|
| RNA (1 μg) | 3.5 | μl |
| Random hexamers (10 μM) | 1 | μl |
| Deionised water | 2.5 | μl |

The contents were mixed and spun briefly in a microfuge.
The samples were incubated at 70° C. for 3 minutes to denature the RNA.
While the RNA was being denatured the following master mix was prepared:

| | Per rxn |
|---|---|
| 5x first strand buffer | 2 μl |
| DTT (20 mM) | 1 μl |
| 50x dNTP mix | 1 μl |
| Total volume | 4 μl |

Tubes were removed from the PCR machine and cooled on ice for 2 minutes then spun briefly to collect contents.
Samples were then incubated @ 42° C. for 2 minutes.
0.5 μl of Powerscript Reverse Transcriptase was added per reaction (1.75 μl) and the master mix mixed well by pipetting.

4.5 μl of the complete master mix was added to each sample and control tube and the samples then incubated @ 42° C. for 60 minutes then the samples transferred to ice.

40 μl of 10 mM Tris/1 mM EDTA pH 7.6 was added to each sample.
The tubes were heated 72° C. for 7 minutes then stored @−70° C. until required.

### PCR Amplification

PCR amplification was performed on 1 μl of bisulphite treated RNA, PCR amplifications were performed in 25 μl reaction mixtures containing 1 μl of bisulphite-treated genomic DNA, using the Promega PCR master mix, 6 ng/μl of each of the primers.

One μl of 1^{st} round amplification was transferred to the second round amplification reaction mixtures. Samples of PCR products were amplified in a ThermoHybaid PX2 thermal cycler under the conditions described in Clarke et al.

TABLE 2
Primers used for the
amplification of the Actin gene

| Gene | Primer Sequence | SEQ ID NO: |
|---|---|---|
| ACTIN-3F1 | gacggccaggtcatcaccattggcaat | 3 |
| ACTIN-3F2 | gttccgctgccctgaggcactcttccag | 4 |
| ACTIN-4R1 | ggtagtttcgtggatgccacaggactc | 5 |
| ACTIN-4R2 | gatgtccacgtcacacttcatgatgga | 6 |
| ACTIN-4F1 | ccatgtaccctggcattgccgacag | 7 |
| ACTIN-4F2 | aggagatcactgccctggcacccagcac | 8 |

Agarose gels (2%) were prepared in 1% TAE containing 1 drop ethidium bromide (CLP #5450) per 50 ml of agarose. Five μl of the PCR derived product was mixed with 1 μl of 5× agarose loading buffer and electrophoresed at 125 mA in X1 TAE using a submarine horizontal electrophoresis tank. Markers were the low 100-1000 by type. Gels were visualised under UV irradiation using the Kodak UVIdoc EDAS 290 system.

### Detection System Using Beads

### Coating Magnetic Beads

The INA used for attachment to the magnetic beads can be modified in a number of ways. In this example, the INA contained either a 5′ or 3′ amino group for the covalent attachment of the INA to the beads using a hetero-bifunctional linker such as EDC. However, the INA can also be modified with 5′ groups such as biotin which can then be passively attached to magnetic beads modified with avidin or steptavidin groups.

Ten μl of carboxylate modified Magnabind™ beads (Pierce) or 100 μl of Dynabeads™ Streptavidin (Dynal) were transferred to a clean 1.5 ml tube and 90 μl of PBS solution added.

The beads were mixed then magnetised and the supernatant discarded. The beads were washed x2 in 100 μl of PBS per wash and finally resuspended in 90 μl of 50 mM MES buffer pH 4.5 or another buffer as determined by the manufactures' specifications.

One μl of 250 μM INA (concentration dependant on the specific activity of the selected INA as determined by oligo hybridisation experiments) is added to the sample and the tube vortexed and left at room temperature for 10-20 minutes.

Ten μl of a freshly prepared 10 mg/ml EDC solution (Pierce/Sigma) is then added, the sample vortexed and incubated at either room temperature or 4° C. for up to 60 minutes.

The samples were then magnetised, the supernatant discarded and the beads may be blocked by the addition of 100 μl either 0.25 M NaOH or 0.5 M Tris pH 8.0 for 10 minutes.

The beads were then washed x2 with PBS solution and finally resuspended in 100 μl PBS solution.

### Hybridisation Using the Magnetic Beads

Ten μl of INA coated Magnabind™ beads were transferred to a clean tube and 40 μl of either ExpressHyb™ buffer (Clontech) either neat or diluted 1:1 in distilled water or Ultrahyb™ buffer (Ambion) either neat or diluted 1:1/1:2 or 1:4 in distilled water added or an in house hybridisation buffer. The buffers may also contain either cationic/anionic or zwittergents at known concentration or other additives such as Heparin and poly amino acids.

Sample RNA 1-5 μl was then added to the above solution and the tubes vortexed and then incubated at 55° C. or another temperature depending on the melting temperature of the chosen INA/RNA hybrid for 20-60 minutes.

The samples were magnetised and the supernatant discarded and the beads washed ×2 with 0.1×SSC/0.1% SDS at the hybridisation temperature from earlier step for 5 minutes per wash, magnetising the samples between washes.

### Preparation of Radio-Labelled Detector Spheres

A INA or oligo molecule can be either 3′ or 5′ labelled with a molecule such as an amine group, thiol group or biotin.

The labelled molecule can also have a second label such as P³² or I¹²⁵ incorporated at the opposite end of the molecule to the first label.

This dual labelled detector molecule can now be covalently coupled to a carboxylate or modified latex bead of known size using a hetero-bifunctional linker such as EDC.

The unbound molecules can then be removed by washing leaving a bead coated with large numbers of specific detector/signal amplification molecules.

These beads can then be hybridised with the nucleic acid sample of interest to produce signal amplification.

### Preparation of Fluorescent Labelled Detector Spheres

An INA or oligo molecule can be either 3′ or 5′ labelled with a molecule such as an amine group, thiol group or biotin.

The labelled molecule can also have a second label such as Cy-3, Cy-5, FAM, HEX, TET, TAMRA or any other suitable fluorescent molecule incorporated at the opposite end of the molecule to the first label.

This dual labelled detector molecule can now be covalently coupled to a carboxylate or modified latex bead of known size using a hetero-bifunctional linker such as EDC.

The unbound molecules can then be removed by washing, leaving a bead coated with large numbers of specific detector/signal amplification molecules.

These beads can then be hybridised with the RNA sample of interest to produce signal amplification.

### Preparation of Enzyme Labelled Detector Spheres

An INA or oligo molecule can be either 3′ or 5′ labelled with a molecule such as an amine group or a thiol group.

The labelled molecule can also have a second label such as biotin or other molecules such as horse-radish peroxidase or alkaline phosphatase conjugated on via a hetero-bifunctional linker at the opposite end of the molecule to the first label.

This dual labelled detector molecule can now be covalently coupled to a carboxylate or modified latex bead of known size using a hetero-bifunctional linker such as EDC.

The unbound molecules can then be removed by washing leaving a bead coated with large numbers of specific detector/signal amplification molecules.

These beads can then be hybridised with the nucleic acid sample of interest to produce signal amplification.

Signal amplification can then be achieved by binding of a molecule such as Streptavidin or an enzymatic reaction involving a colorimetric substrate.

### INA Oligomer Combinations

The initial hybridization event preferably involves the use of magnetic beads coated with a INA complimentary to the RNA of interest.

A second hybridisation event, if required, can involve any of the detection methods mentioned above.

This hybridisation reaction can be done with either a second INA complimentary to the nucleic acid of interest or an oligo or modified oligo complementary to the RNA of interest.

### Dendrimers and Aptamers

Dendrimers are branched tree-like molecules that can be chemically synthesised in a controlled manner so that multiple layers can be generated that were labelled with specific molecules. They were synthesised stepwise from the centre to the periphery or visa-versa.

One of the most important parameters governing dendrimer structure and its generation is the number of branches generated at each step; this determines the number of repetitive steps required to build the desired molecule.

Dendrimers can be synthesised that contain radioactive labels such as 1¹²⁵ or P³² or fluorescent labels such as Cy-3, Cy-5, FAM, HEX, TET, TAMRA or any other suitable fluorescent molecule to enhance signal amplification.

Alternatively dendrimers can be synthesised to contain carboxylate groups or any other reactive group that could be used to attach a modified INA or DNA molecule.

### Detection System Using Arrays

Treated RNA can be applied to any suitable substrate to form arrays such as microarrays that can be screened for activity of genes or expression units of interest. Persons skilled in the art would be familiar with the appropriate technology for making suitable arrays.

### EXAMPLES

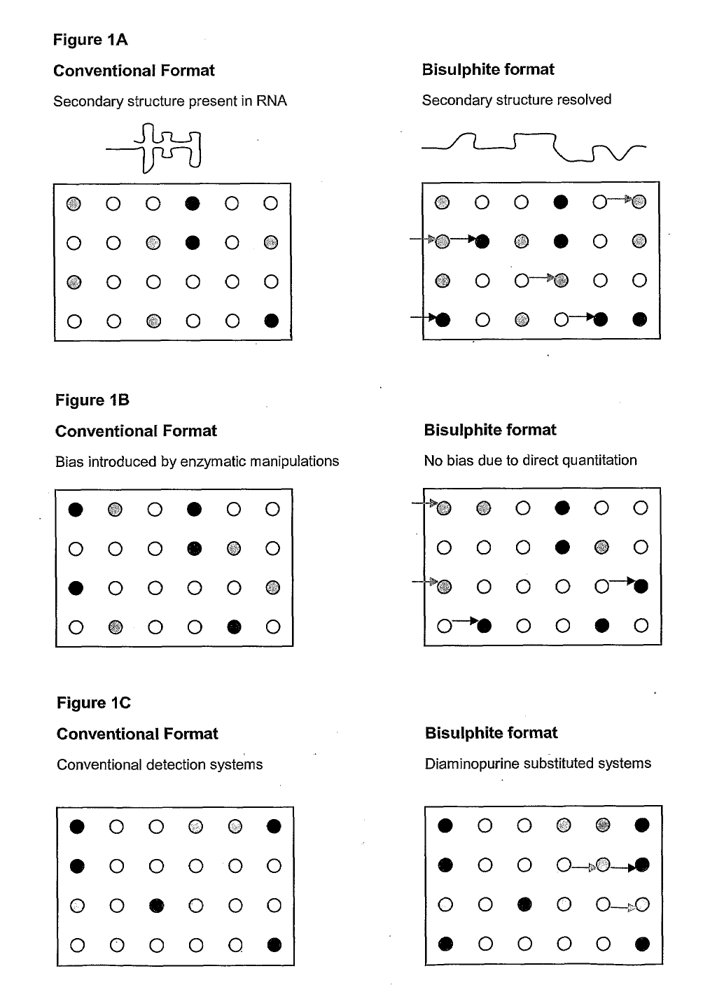

FIG. 1A, FIG. 1B and FIG. 1C show the comparison of the present invention (bisulphite treated RNA) with prior art using a typical micro-array based assay. FIG. 1A shows a representation of a typical micro-array based assay where dark spots indicate genes that are up-regulated in a certain RNA population and light spots indicate down-regulated genes within the same population. Dark and light arrows indicate genes that are detected as being up-regulated and down-regulated respectively in the bisulphite treated RNA but not in conventional methods due to secondary structure which prevents the detector molecules in the conventional system from binding to their target.

FIG. 1B shows a micro-array based assay similar to FIG. 1A but dark and light arrows indicate genes that are detected as being up-regulated and down-regulated respectively in the bisulphite treated RNA but not in conventional methods due to bias produced during enzymatic manipulation of the RNA prior to expression analysis, resulting in incorrect determination of RNA expression levels. Enzymatic manipulations can cause misleading results and indicate that certain genes are up or down regulated when in fact they are not up or down regulated.

FIG. 1C shows a micro-array based assay similar to FIG. 1A but dark and light arrows indicate genes that are detected as being up-regulated and down-regulated respectively in the bisulphite treated RNA but not in conventional methods due to improved specificity of the detector molecules. Increasing the specific binding strength of the detector molecules leads to the detection of RNA species which may not be detected using conventional methods due to lack of specificity.

### Actin

RNA was extracted and purified then bisulphite treated and amplified as already described. After amplification PCR products were purified using the Marligen PCR clean up kit as instructed by the manufactures and resuspended in 20 μl of water. 100 ng of reverse primer was added to 10 μl of PCR product and the samples sent to Supermac (Camperdown, Sydney) for DNA sequencing.

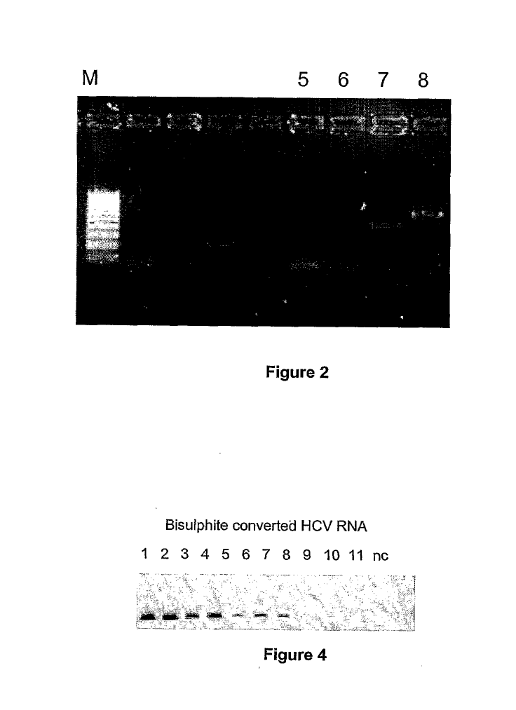

FIG. 2 shows reverse transcriptase PCR using bisulphite modified total RNA from cell line material. As can be seen from the Figure when wild type primers (non-bisulphite converted) directed to exon 3a-4 or exon 3a-3b of the Actin gene are used no amplification product is obtained in either case. This indicates that the conversion of RNA is very efficient as no wild type sequences can be detected. Conversely, if bisulphite converted primers are used on the same sample to exon 3a-4 or exon 3a-3b distinct PCR bands are generated indicating that it is possible to amplify specific mRNA's from bisulphite converted material.

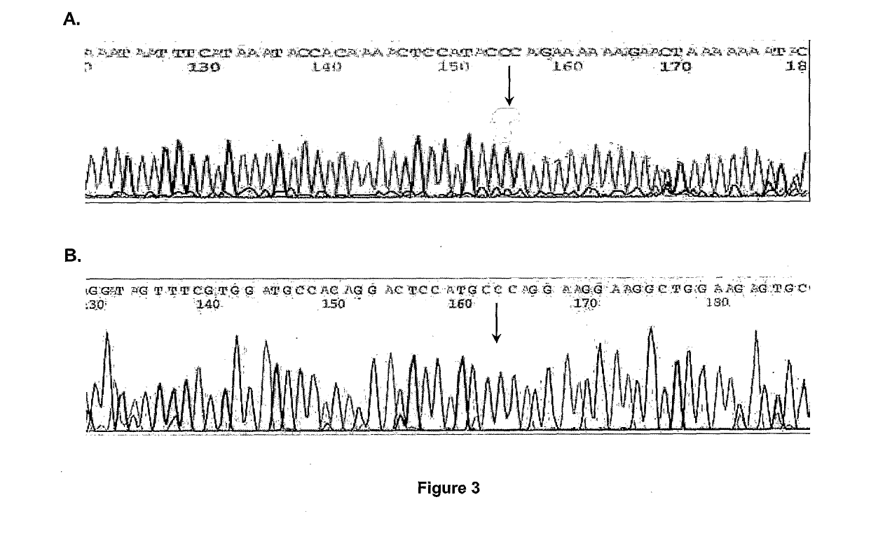

FIG. 3 shows direct sequencing of the PCR products generated from the bisulphite converted RNA. As can be seen from the sequencing profile, the PCR products are derived from the RNA, not contaminating DNA as the sequence of the products carry on over the splice site between exons 3 and 4. In addition as can be seen from FIG. 3 the PCR products have been fully converted as all the original C residues in the sample are now converted to T residues:

### Hepatitis C Virus

Hepatitis C virus (HCV) RNA samples were obtained from Acrometrix (OptiQual HCV high positive control) or BBI diagnostics (HCV RNA linearity panel) and purified with Ultrasens Viral purification kit according to the manufacturer's instructions. Samples were treated with sodium bisulphite and converted HCV RNA samples were reverse transcribed with Superscript III reverse transcriptase (Invitrogen) as follows:

11 μl converted RNA template

1 μl random primer (300 ng/μl)

1 μl dNTPs (10 mM)

Samples were heated at 65° C. for 5 minutes, then placed immediately on ice for at least one minute, after which the following reagents were added:

4 μl 5× First strand buffer

1 μl RNase OUT (40 U/μl)

1 μl DTT (100 mM)

1 μl Superscript III (200 U/μl)

The samples were reverse transcribed using the following conditions:

25° C., 12 minutes

27° C., 2 minutes

29° C., 2 minutes

31° C., 2 minutes

33° C., 2 minutes

35° C., 2 minutes

37° C., 30 minutes

45° C., 15 minutes

50° C., 5 minutes

75° C., 5 minutes

Two μl of cDNA was then PCR amplified with primers and probe specific for the 5′ NTR of HCV:

(Forward primer—ttatgtagaaagtgtttagttatggtgt (SEQ ID NO: 9);
Reverse primer—acccaaatytccaaayattaaacaaat (SEQ ID NO: 10);
Probe—tcCacAaaCcaCtaTaaCtcTcc (SEQ ID NO: 11),
(where capital letters indicate the presence of LNAs) using the following reagents and cycling conditions in a Corbett 6000 Rotor Gene:

| | | |
|---|---|---|
| Sigma Jumpstart 2x master mix | 12.5 | μl |
| Forward primer | 50 | ng |
| Reverse primer | 50 | ng |
| 25 mM MgCl₂ | 3.5 | μl |
| 400 nM Probe (final concentration) | x | μl |
| Water up to 23 μl | x | μl |
| 95° C., 10 minutes | | |
| 95° C., 10 seconds (50x) | | |
| 53° C., 90 seconds (50x) | | |
| 60° C., 30 seconds | | |

Results are shown in FIG. 4 where the detection limit for the assay as can be seen from the figure is around 2.5 IU of virus.

TABLE 3
Summarises the results shown in FIG. 4

| Sample | IU/ | Copies/ | | IU/ | Copies/ |
|---|---|---|---|---|---|
| No | PCR | PCR | Sample# | PCR | PCR |
| 1 | 1.2 × 10⁵ | 3.2 × 10⁵ | 7 | 3.8 | 10 |
| 2 | 2 × 10⁴ | 5.3 × 10⁴ | 8 | 1 | 2.7 |
| 3 | 1.2 × 10⁴ | 3.2 × 10⁴ | 9 | 0.3 | 1 |
| 4 | 2.5 × 10³ | 6.7 × 10³ | 10 | 0 | 0 |
| 5 | 1.4 × 10² | 3.7 × 10² | 11 | 0 | 0 |
| 6 | 11.8 | 31 | | | |

The gel results shown in FIG. 4 indicate that the bisulphite method can be used to monitor expression levels of the HCV over a wide range of target concentrations down to as low as 2.5 IU of HCV.

nc=negative control

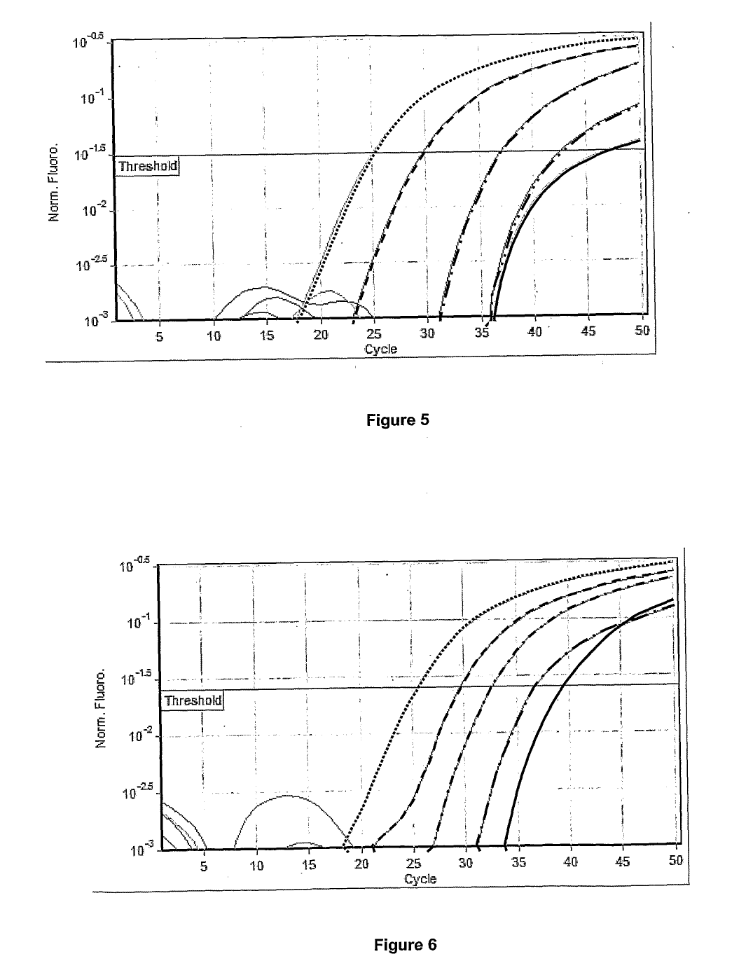

Real time qPCR results of a linearity panel titration for HCV are shown in FIG. 5. The Standard Curve for this plot was linear having R Value of 0.99947 and R̂2 Value of 0.9989.

TABLE 4
Samples of treated HPV analysed in FIG. 5.

| Sample | | | Given | Calc | |
|---|---|---|---|---|---|
| No | Line | Ct | Conc (IU) | Conc (IU) | Var (%) |
| 1 | ........ | 25.06 | 3,250.0 | 3,591.8 | 10.5 |
| 2 | ----- | 29.75 | 732.5 | 670.0 | 8.5 |
| 3 | . | 37.03 | 55.0 | 49.5 | 10.0 |
| 4 | .. | 42.60 | 6.0 | 6.8 | 12.6 |
| 5 | | 46.87 | 1.5 | 1.5 | 2.4 |

Real time qPCR results of quantitation report over dynamic range titration for HCV is shown in FIG. 6. The Standard Curve for this plot was linear having R Value of 0.99856 and R̂2 Value of 0.99713.

TABLE 5
Samples of treated HCV analysed in FIG. 6

| Sample | | | Given | Calc | |
|---|---|---|---|---|---|
| No | Line | Ct | Conc (IU) | Conc (IU) | % Var |
| 1 | ........... | 25.53 | 156,250.0 | 169,174.2 | 8.3 |
| 2 | ------ | 29.68 | 31,250.0 | 26,876.3 | 14.0 |
| 3 | . . . | 32.67 | 6,250.0 | 7,117.9 | 13.9 |
| 4 | . | 36.89 | 1,250.0 | 1,090.6 | 12.8 |
| 5 | | 39.53 | 312.5 | 337.7 | 8.1 |

The results from the linearity panel and the dynamic range samples show the quantitation curves generated during real time PCR. The point at which the line of the curve crosses the threshold is known as the Ct value and is used for quantitation of the samples. A series of known concentrations of virus, over 3 orders of magnitude for each set of samples, were purified, bisulphite converted and amplified and the standard curves generated show that the reaction efficiencies are constant and linear over the range of concentrations examined, as exemplified by the R² value being close to 1. These results and those in FIG. 4 demonstrate that there is good sensitivity and specificity for the detection of HCV viral RNA, using end-point PCR and real time PCR using a viral-specific probe, ranging from 156250 IU down to 1.5 IU, illustrating that the assay can detect expression of viral genes over a very broad range of concentrations.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1-15. (canceled)

16. An assay for gene expression comprising:
treating messenger RNA (mRNA) with an agent selected from the group consisting of sodium bisulphite, sodium metabisulphite and guanidinium hydrogen sulphite under conditions to substantially remove secondary structure of the mRNA; and
measuring the presence or amount of treated mRNA using probes that contain bases A (adenine), T (thymine) and C (cytosine) and are substantially free of G (guanine) so as to obtain an indication of gene expression.

17. The assay according to claim 16 wherein the probes are selected from the group consisting of oligonucleotide, PNA, LNA and INA probes.

18. The assay according to claim 17 wherein the probe is an INA probe.

19. The assay according to claim 16 wherein the mRNA is obtained from an animal, plant, microorganism, cell, cells or a cell population.

20. The assay according to claim 19 wherein the microorganism is a virus or bacterium.

21. The assay according to claim 19 wherein the animal is a human.

22. The assay according to claim 1 wherein the agent is sodium metabisulphite.

23. An assay for gene expression comprising:
treating mRNA with an agent selected from the group consisting of sodium bisulphite, sodium metabisulphite and guanidinium hydrogen sulphite under conditions to substantially remove secondary structure of the mRNA;
reverse transcribing and amplifying the mRNA using primers capable of binding to complementary sequences of mRNA, wherein the primers contain bases A (adenine), T (thymine) and C (cytosine) and are substantially free of G (guanine); and
measuring the presence or amount of treated and amplified mRNA so as to obtain an indication of gene expression.

24. The assay according to claim 23 wherein the mRNA is obtained from an animal, plant, microorganism, cell, cells or a cell population.

25. The assay according to claim 24 wherein the microorganism is a virus or bacterium.

26. The assay according to claim 24 wherein the animal is a human.

27. The assay according to claim 23 wherein the agent is sodium metabisulphite.


SEQUENCE LISTING (11 sequences)

SEQ ID NO: 1 - DNA, 59 bp, Homo sapiens
aaataatttcataaataccacaaaactccatacccaagaaaaaagaactaaaaaaatac

SEQ ID NO: 2 - DNA, 58 bp, Homo sapiens
ggatagtttcgtggatgccacaggactccatgcccaggaaggaaggctggaagagtgc

SEQ ID NO: 3 - DNA, 27 bp, Homo sapiens
gacggccaggtcatcaccattggcaat

SEQ ID NO: 4 - DNA, 28 bp, Homo sapiens
gttccgctgccctgaggcactcttccag

SEQ ID NO: 5 - DNA, 27 bp, Homo sapiens
ggtagtttcgtggatgccacaggactc

SEQ ID NO: 6 - DNA, 27 bp, Homo sapiens
gatgtccacgtcacacttcatgatgga

SEQ ID NO: 7 - DNA, 25 bp, Homo sapiens
ccatgtaccctggcattgccgacag

SEQ ID NO: 8 - DNA, 28 bp, Homo sapiens
aggagatcactgccctggcacccagcac

SEQ ID NO: 9 - DNA, 28 bp, Hepatitis C
ttatgtagaaagtgtttagttatggtgt

SEQ ID NO: 10 - DNA, 27 bp, Hepatitis C
acccaaatytccaaayattaaacaaat

SEQ ID NO: 11 - DNA, 23 bp, Hepatitis C
tccacaaaccactataactctcc